(19) United States
(12) Patent Application Publication
(10) Pub. No.: US 2012/0071837 A1
(43) Pub. Date: Mar. 22, 2012

(54) AUTO INJECTOR FOR MEDICATION

(76) Inventors: Sean O'Connor, West Chester, PA (US); Luis Jahn, Royersford, PA (US)

(21) Appl. No.: 13/233,525

(22) Filed: Sep. 15, 2011

Related U.S. Application Data
(60) Provisional application No. 61/384,871, filed on Sep. 21, 2010.

(51) Int. Cl. A61M 5/315

(52) U.S. Cl. 604/232

(57) ABSTRACT

Described is drug infusion device. The device is intended to permit the delivery of medication over an extended period of time.

### CROSS REFERENCE TO RELATED APPLICATIONS

This application relates to U.S. patent application Ser. No. 61/384,871, filed Sep. 21, 2010; all applications are herein incorporated by reference in their entireties.

### FIELD OF THE INVENTION

The present invention relates, in general, to drug delivery devices and, more particularly, to auto-injection devices for medications.

### BACKGROUND OF THE INVENTION

The use of drug delivery devices for various types of drug therapy is becoming more common as the automated infusion of a drug may provide more reliable and more precise treatment to a patient.

Diabetes is a major health concern, as it can significantly impede on the freedom of action and lifestyle of persons afflicted with this disease. Typically, treatment of the more severe form of the condition, Type I (insulin-dependent) diabetes, requires one or more insulin injections per day, referred to as multiple daily injections. Insulin is required to control glucose or sugar in the blood, thereby preventing hyperglycemia that, if left uncorrected, can lead to ketosis. Additionally, improper administration of insulin therapy can result in hypoglycemic episodes, which can cause coma and death. Hyperglycemia in diabetics has been correlated with several long-term effects of diabetes, such as heart disease, atherosclerosis, blindness, stroke, hypertension, and kidney failure.

The value of frequent monitoring of blood glucose as a means to avoid or at least minimize the complications of Type I diabetes is well established. Patients with Type II (non-insulin-dependent) diabetes can also benefit from blood glucose monitoring in the control of their condition by way of diet and exercise. Thus, careful monitoring of blood glucose levels and the ability to accurately and conveniently infuse insulin into the body in a timely manner is a critical component in diabetes care and treatment.

To more effectively control diabetes in a manner that reduces the limitations imposed by this disease on the lifestyle of the affected person, various devices for facilitating blood glucose (BG) monitoring have been introduced. Typically, such devices, or meters, permit the patient to quickly, and with a minimal amount of physical discomfort, obtain a sample of their blood or interstitial fluid that is then analyzed by the meter. In most cases, the meter has a display screen that shows the BG reading for the patient. The patient may then dose theirselves with the appropriate amount, or bolus, of insulin. For many diabetics, this results in having to receive multiple daily injections of insulin. In many cases, these injections are self-administered.

Due to the debilitating effects that abnormal BG levels can have on patients, i.e., hyperglycemia, persons experiencing certain symptoms of diabetes may not be in a situation where they can safely and accurately self-administer a bolus of insulin. Moreover, persons with active lifestyles find it extremely inconvenient and imposing to have to use multiple daily injections of insulin to control their blood sugar levels, as this may interfere or prohibit their ability to engage in certain activities. For others with diabetes, multiple daily injections may simply not be the most effective means for controlling their BG levels. Thus, to further improve both accuracy and convenience for the patient, insulin infusion pumps have been developed.

Insulin pumps are generally devices that are worn on the patient's body, either above or below their clothing. Because the pumps are worn on the patient's body, a small and unobtrusive device is desirable. Therefore, it would be desirable for patients to have a more compact drug delivery device that delivers medication reliably and accurately. Further, it would be desirable for such devices to provide improved performance, ease of use, and reduced pain to those requiring large quantities of injected medications.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an exemplary embodiment of an auto-injector device in perspective, cutaway view.

FIG. 2 illustrates another exemplary embodiment of an auto-injector device according to the present invention in three instances as the amount of fluid in the drug reservoir is reduced.

### DETAILED DESCRIPTION OF ILLUSTRATIVE EMBODIMENTS OF THE INVENTION

The invention relates to an injection device, also called an auto-injector, for delivering medication to a patient. For many years, patients have been given medication via needles and syringes. In instances where large quantities of medication must be delivered over an extended period of time, intravenous “drips” have been used, but such devices typically require a trained healthcare provider, such as a nurse or doctor to ensure that the infusion device is correctly inserted through the skin of the patient. Further, the medication is often mixed with a saline solution for delivery to the patient over a period of hours or longer.

Presently, inadequate devices exist to permit individuals, such as insulin-dependent diabetics, to safely self-administer the often large quantity of medication that they require on a daily or multiple-daily basis. Typically, such diabetics must inject their medication via a syringe over a period of 10-30 minutes (or longer) using a needle that pierces their dermal tissue. Often, due to the pain associated with these injections, the patient will attempt to “rush” delivery of their medication, further increasing their pain and forcing their medication to disperse through the dermal tissue due to the large size of insulin molecules and the high viscosity of the drug. Moreover, it is often beneficial from a therapeutic standpoint for insulin dependent diabetics to receive their injections over an extended period of time.

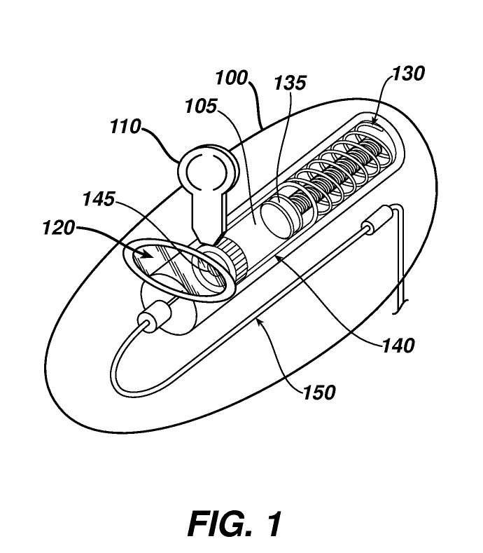

Given this need, it has been found that a device that is capable of storing a large quantity of medication and delivering it over an extended period of time can be therapeutically beneficial to patients with many afflictions, particularly diabetes. As shown in FIG. 1, an exemplary structure for such a device many comprise, consist of, or consist essentially of a housing 100 that has a cavity 105 therein. Within the cavity 105 may be disposed a cartridge 140 containing a quantity of medication. At one end, the cartridge has a septum 145 for receiving an infusion device 150. At the other end, the cartridge 140 receives a plunger 135 that is biased by one or more springs 130.

Those skilled in the art will recognize that the springs may be replaced by worm drives, lead screws, or other devices that can be configured to elongate at a predetermined rate, to bias the plunger 135 into the cartridge 140 to eject medication via the septum 145 and through the infusion device 150 and ultimately into the patient. When springs are used, it is desirable to minimize the length of the cartridge 140 to maintain substantially linear spring force travel. While small variations in spring force will not substantially negatively affect the delivery of medication, it is desirable that the rate of drug delivery be maintained as substantially constant until the cartridge is empty.

The device may also contain an activation tab 110 that separates the septum 145 from the infusion device 150. Pulling the activation tab 110 allows the infusion device 150 to pierce the septum 145 and begin drug delivery into the patient. Once the drug reservoir in the cartridge is exhausted, the device may be discarded. Alternatively, the device may include means for inserting a full cartridge or refilling the cartridge. A sight window 120 may be provided to allow the user to visually determine whether drug remains in the cartridge. As well, although not shown in the figures, another spring or set of springs may be included to bias the infusion set through the septum 145 when the activation tab 110 is removed.

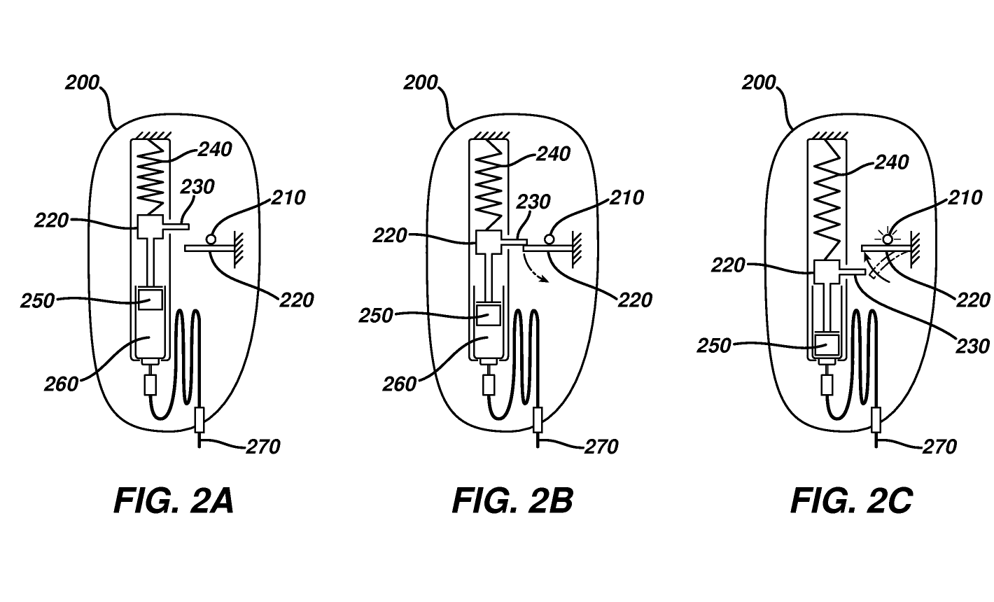

FIG. 2 illustrates another embodiment of the invention in which the plunger 220 has a protrusion 230. The interior of the housing 200 includes a post 210 placed adjacent to a leaf spring 220 or similarly flexible member (which could be as simple as a soft plastic protrusion). As the plunger 220 moves as a result of the biasing of the one or more springs 240, the protrusion 230 nears the leaf spring 220. As the cartridge nears exhaustion, the protrusion 230 biases the leaf spring 220 and when the cartridge is empty (or drug delivery is complete), the protrusion 230 passes the leaf spring 220, causing the leaf spring 220 to recoil and impact against the post 210. The concussive force of the impact causes an audible “click” to indicate to the user that drug delivery is complete and the device may be removed.

It will be recognized that equivalent structures may be substituted for the structures illustrated and described herein and that the described embodiment of the invention is not the only structure, which may be employed to implement the claimed invention. In addition, it should be understood that every structure described above has a function and such structure can be referred to as a means for performing that function. While embodiments of the present invention have been shown and described herein, it will be obvious to those skilled in the art that such embodiments are provided by way of example only. Numerous variations, changes, and substitutions will now occur to those skilled in the art without departing from the invention.

Typically, the size of the cartridge will be determined by the amount of medication that the patient requires with each injection. With insulin, typical cartridges may range from 50 units (0.5 ml) to 300 units (3 ml). The spring force necessary to deliver the medication via the infusion device is dependent on the viscosity of the drug in the cartridge and the desired delivery time. For example, a viscous drug delivered over a short period of time will require a spring system with higher force than a relatively low viscosity drug to be delivered over a long period of time.

It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A device for injecting medication, comprising:
a housing;
a cavity configured to accept a drug cartridge;
a plunger configured to interact with the drug cartridge;
at least one biasing spring biased against an inner surface of the cavity and the plunger; and
an infusion device configured to carry fluid from the drug cartridge to a catheter.
